(19) United States
(12) Patent Application Publication
(10) Pub. No.: US 2024/0225714 A1
(43) Pub. Date: Jul. 11, 2024

(54) METHOD AND APPARATUS FOR HIGH-POWER ABLATION

(71) Applicant: Yakov NEDLIN, Hermesh (IL)

(72) Inventors: Yakov NEDLIN, Hermesh (IL); Gregory GOLOVCHINER, Petah Tikva (IL)

(73) Assignee: Yakov NEDLIN, 44 Hermesh (IL)

(21) Appl. No.: 18/287,473

(22) PCT Filed: Apr. 20, 2022
(86) PCT No.: PCT/IL2022/050410

§ 371 Date: Oct. 19, 2023

Related U.S. Application Data
(60) Provisional application No. 63/177,402, filed on Apr. 21, 2021.

(51) Int. Cl. A61B 18/02; A61B 18/00

(52) U.S. Cl. CPC A61B 18/0218; A61B 2018/00577; A61B 2018/00702; A61B 2018/0212

(57) ABSTRACT

Apparatus for cryoablation comprises a super-cooled liquid nitrogen streaming mechanism and an adjustable area-targeting cryoablation tip for use at a cryoablation site. The use of super-cooled liquid nitrogen increases the power and, thus, the cryoablation lesion depth.

### RELATED APPLICATION

This application claims the benefit of priority of U.S. Provisional Patent Application No. 63/177,402 filed 21 Apr. 2021, the contents of which are incorporated herein by reference in their entirety.

### FIELD AND BACKGROUND OF THE INVENTION

The present invention, in some embodiments thereof, relates to a method and apparatus for tissue ablation and, more particularly, but not exclusively, to a high-power cryoablation system for treatment of cardiac arrhythmia and any other condition requiring tissue ablation. Additional indications discussed herein technology are neural tumors, cancer tumors, fibroids, and other volumetric substrates.

Currently practiced treatments for heart arrhythmias are Antiarrhythmic Medications, Implantable Defibrillators, Pacemakers or Resynchronizators, Ablations and Surgery (Maze procedure).

The present disclosure relates to ablation. Doctors typically perform cryoablation as a treatment for Atrial fibrillation, Atrial flutter, Supraventricular tachycardia (SVT), Atrial tachycardia, Ventricular fibrillation, and Ventricular tachycardia. The treatment restores normal heart rhythm by disabling heart cells that disturb the spread of the electrical signal through the heart and thus create an irregular heartbeat. The procedure is minimally invasive and involves inserting of a thin flexible tube called a catheter to locate and either heat or freeze the heart tissue that triggers an irregular heartbeat.

Existing solutions for ablation include heat ablation, cryoablation and electroporation.

Heat ablation is performed by cardiac catheterization and relies on technologies such as Radiofrequency, Laser, Concentrated US, Microwave, etc. The solution indications are shallow tachycardias, Atrial Fibrillation, SVT, etc. The clinical procedure involves catheterization, mapping for substrate tissue surveillance, navigation to the treatment area, ablation involving heat application, cooling, and extraction.

Heat ablation is limited in its power by a physiological barrier. Applying higher power, which is needed to perform transmural, that is through the whole heart wall thickness, ablation, will nevertheless result in tissue overheating and vapor bubble formation that may subsequently cause dangerous clinical situations such as endocardial rupture, clots and more. Any heat ablation catheter, no matter what technology it relies on, is limited to about 60° C. at its tip by these safety aspects.

Using cold, rather than heat, to disable arrhythmic tissue reduces risks of acute and sequential damage to the treated tissue (e.g. hypertrophic fibrosis, clots) and collateral damage to surrounding structures (e.g. esophageal injury). In addition, many studies show that patients, treated by cryoablation, experience less pain, and recover faster than patients treated by heat ablation.

Thus, a successful cryoablation system may be able to perform transmural ablation to treat deep arrhythmic substrates (tissues) with none of the above-mentioned risks.

Accordingly, there are several cryoablation technologies currently available, one of which is in widespread use and others are in trial.

The most common cryoablation technology is Nitrous Oxide Evaporation which is widely in use in cryoballoons and indicates mostly Atrial Arrhythmias. Focal point catheters based on this technology may be used in precise ablations in atria or rarely in shallow epicardial ventricular arrhythmia ablations. This technology is limited in its' power by the physical property of the coolant and achieves lowest temperatures at −88° C. in an artificial and adiabatic environment but in practice operates at much higher temperatures during actual procedures in patients.

Another cardiac cryoablation technology relies on the Joule-Thomson cooling effect. Systems using this technology require a supply of a very high pressurized gas, that acts as a coolant, normally Argon.

Drawbacks of Joule-Thomson based systems include the limited ablating power, which is prescribed by the physical parameters of the coolant such as pressure, flow rate and the Joule-Thomson coefficient of the coolant. Argon is considered as one of the most suitable and effective substances for this technology and it achieves −186° C. in an artificial and adiabatic environment although again, the actual operating temperature with patients is higher, thus limiting the effectiveness of this technology in performing transmural epicardial ablations.

The third cooling technology is supercritical or near-critical nitrogen cardiac cryoablation. Supercritical nitrogen cryoablation uses pressurized and precooled gaseous nitrogen. The solution provides higher power to the treated area, compared to Joule-Thomson, but is also energy-limited, as its' lowest achievable temperature is about −196° C. but again only in an artificial and adiabatic environment. Actual temperature at the point of care depends on the system's efficiency and insulation rate and may not be sufficient for deep ventricular epicardial ablations.

Additional prior art is as follows:
1. US2019076179(A1)—Ablation Catheter Having A Shape Memory Stylet, which discloses different tips that may be used.
2. U.S. Pat. No. 6,106,518(A)—discloses a variable geometry tip for a cryosurgical ablation device.
3. US2012283722(A1)—discloses an adiabatic cooling system for medical use.

However, there are certain types of arrhythmia that are deep and are not susceptible to the above methods. Higher power cryoablation is required to treat deep myocardial electro-conduction disorders such as Ventricular tachycardia. Existing cryoablation devices aim at shallow arrhythmias, and an ability to aim at and reach and affect cells at depth is needed.

### SUMMARY OF THE INVENTION

The present embodiments may utilize super-cooled liquid nitrogen (SCLN2) to provide a high-power solution for previously non-treatable or less easily treatable cardiac arrhythmias, neural tumors, cancer tumors, fibroids, and other volumetric substrates. The use of boiling liquid makes available the latent heat (enthalpy of boiling liquid) of liquid nitrogen as an energy source—or more accurately an energy sink—to make the treatment more effective.

According to an aspect of some embodiments of the present invention, there is provided apparatus for cryoablation comprising:
a supply unit configured to provide super-cooled liquid nitrogen for streaming; and
an adjustable cryoablation tip for use at a cryoablation site, namely the point of cure. The tip may receive the super-cooled or boiling liquid nitrogen and may exchange heat using the liquid nitrogen, thereby to carry out ablation.

In an embodiment, the supply unit comprises a well-insulated pressure tank and a liquid nitrogen tank. The pressure tank may be connected to the liquid nitrogen tank to supply gaseous cool nitrogen and may be controlled to provide a pressure to maintain the liquid nitrogen as a liquid at boiling point or below, thereby to take advantage of latent heat to prevent changes of temperature of the liquid nitrogen.

Embodiments may comprise catheter tubing to connect between the tip and the liquid nitrogen tank, the catheter tubing comprising an inner tube and a concentrically located outer tube, the inner tube for supplying liquid nitrogen to the tip and the outer tube for carrying exhaust and for insulating the inner tube. The boiling liquid in the exhaust tube has a lower pressure than in the inner tube, thus its temperature may be even lower (cooler), and in such a way the outer tube not only insulates the coolant in the inner tube but may even cool it down. The third layer outwards of the center line is an insulation layer that may be executed by insulating polymer foam or, as suggested herein, using a vacuum gap. The insulation layer may be blocked at the distal side, just about the tip, at the distal end of the sheath. At the proximal side the vacuum gap may be connected to the console through the pneumatic interface and to the vacuum source. The vacuum source may be a vacuum suction device or the institutional vacuum suction port.

Embodiments may comprise a manifold between the catheter tubing and the tip, the manifold configured to connect the inner tube to provide the liquid nitrogen to an inlet of the tip, and to connect the outer tube to an outlet of the tip.

Embodiments may comprise a sheath, the sheath having a proximal end and a distal end, the distal end containing the tip, the sheath being controllably steerable to enable the distal end to reach a cryoablation site.

In an embodiment, the sheath comprises a closure over the tip, the closure being controllably openable on reaching the cryoablation site to reveal the tip.

In an embodiment, the sheath or the tip may comprise electrodes therefor endocardial surface electrical surveillance.

In an embodiment, the tip comprises a hollow tube, the hollow tube acting as a heat exchanger and having an inlet for the inflow of the liquid nitrogen and an outlet for the outflow of the liquid nitrogen after use as exhaust.

In an embodiment, the hollow tube is arranged in a spiral coil.

In an embodiment, a controllably released tip allows adjusting of the ablation footprint diameter. The adjustment action may use a knob built on the catheter handle. The knob positions may be marked, for example, zero for the folded position, 25 for the fully opened spiral tip with diameter of 25 millimeters.

In an embodiment, the tip is made of a material having a shape memory, e.g., Nitinol.

Embodiments may comprise a controllable release mechanism to release the tip in different sizes for different energy release requirements and different treated area shapes.

Devices according to the present embodiments may control pressure at a tip inlet in order to maintain the liquid nitrogen as a boiling liquid. Alternatively, an embodiment may have a sensor measuring density of the exhaust flow. As far as liquid phase persists in the exhaust tube it may persist also in the tip and the heat exchanging zone.

Embodiments may comprise a vacuum source connected to an outlet of the tip and, in such a way, enlarge flow rate through the tip, in which may provide improved heat transfer and higher ablation power.

Embodiments may comprise control software, for example embedded in the User Interface and/or in the control module, to evaluate ablation depth and the overall chance of a successful procedure, by evaluation of an amount of energy released at the tip, and its comparison to the energy required to change the temperature of the targeted volume of tissue to the desired level, say −20° C., which is considered as the lethal temperature required for ablation.

According to a second aspect of the present invention there is provided a method for cryoablation comprising:
providing super-cooled liquid nitrogen for streaming;
streaming the super-cooled liquid nitrogen to reach a cryoablation tip at lower than boiling temperature for use at a cryoablation site, thereby to carry out ablation. The method may comprise monitoring an amount of energy released at the tip and comparing the amount with energy needed by a defined volume of tissue.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified flow chart of the general procedure for cryoablation according to the known art;

FIG. 2 is a simplified block diagram showing the components of a cryoablation device and a cryoablation catheter according to embodiments of the present invention;

FIG. 3 is a graph of the pressure characteristics of nitrogen showing points that may be used in the present embodiments;

FIG. 4 is the same graph as FIG. 3 showing further points on the nitrogen pressure characteristics that may be used in the present embodiments;

FIG. 5 is a simplified diagram showing a manifold for connecting supply tubes to the tip according to embodiments of the present invention;

FIG. 6 is a simplified diagram showing an exemplary structure for a cryoablation tip as used in embodiments of the present invention;

FIG. 7 is a simplified diagram of the distal end of a sheath with a folded tip exposed;

FIG. 8 is a simplified diagram of the distal end of the sheath as in FIG. 7, with the tip closed inside;

FIG. 9 is a simplified diagram illustrating the pneumatic circuit used in embodiments of the present invention;

FIG. 10 is a simplified diagram showing the control system of the present embodiments overlaid on the pneumatic circuit of FIG. 9;

FIG. 11 is a qualitative diagram of the ablation power ratio to treated tissue volume;

FIG. 12 is a graph of ablation front radius against temperature for the heating case; and

FIG. 13 is a graph of ablation front radius against temperature for the cooling case.

### DESCRIPTION OF SPECIFIC EMBODIMENTS OF THE INVENTION

The present invention, in some embodiments thereof, relates to a method and apparatus for cardiac ablation and, more particularly, but not exclusively, to a high power cardiac cryoablation system. Other embodiments of the invention may be applicable in ablation of neural tumors, cancer tumors, fibroids, and other volumetric substrates.

High Power Cryoablation enables the treatment of disorders that have not yet been ablated, new indications, or disorders which heat ablation or current art cryoablation has not been able to treat or are treating partially. In comparison with common indications such as shallow atrial arrhythmias, a device according to the present embodiments may have all the advantages of a cryoablation system over a heat ablation system, that are safety, reduced post-procedure pain and complications, like stroke, etc.

The higher power of the system of the present embodiments compared to the Joule-Thomson technology and Nitrous Oxide Evaporation technology of the current solution may ensure that the treatment time is reduced, and that the variety of clinical indications is much wider, extending ablation treatment to deep substrates such as ventricular tachycardia (VT).

The present embodiments may provide a cryoablation technology that fits a wider variety of indications, that is cheap and uses a safe coolant, liquid nitrogen, which is cheaper than agents needing pressurized gas tanks and is available in almost all modern hospitals. A predictable lesion volume can be obtained by an evaluation algorithm based on the hardware and the software of the present embodiments, the solution may provide a real time feedback to support clinical decision taking.

That is to say, the present embodiments use a different cooling technology, cold liquid nitrogen streaming carries out the cooling, as opposed to the Joule-Thomson system which uses gas expansion or Nitrous Oxide Evaporation.

The present embodiments have a physical advantage over supercritical nitrogen cryoablation. By using super-cooled liquid nitrogen, the present embodiments may provide higher power to the tip. Super cooling affords an additional energy boost, so the coolant does not boil immediately after pressurizing but rather boils later, on its way to the tip, while latent heat prevents further temperature loss of the fluid once it achieves the boiling point. Hence there is no effective temperature loss at the tip and the actual coolant at the tip has a greater liquid-gas ratio than without super-cooling. By contrast the gaseous nitrogen of the supercritical nitrogen cryoablation system, even if precooled, loses its low temperature all over the tubing and the tip.

It is noted that the coolant is not necessarily at the boiling state when it reaches the tip, although at the outlet of the tip the coolant is at boiling point. Thus, it is possible to keep vapor quality or percentage as low as possible in the heat exchange area so that heat transfer will be most effective. The vapor quality evaluation may be done by mass-rate sensor situated at the exhaust line. Knowing the mass, the pressure and the temperature of the fluid allows vapor quality calculation based on Van Der Waals equation or Nitrogen Saturation tables.

A device according to the present embodiments may exclusively use liquid nitrogen and may have the additional advantage of shortening the required preparation time and procedure expenses.

Again, the present embodiments provide a higher energy level to the tip and thus provide a higher chance of success, especially in the treatment of VT. Unlike supercritical nitrogen cryoablation, the present embodiments may use mass flux sensing to evaluate the real-time evaporation rate of the coolant and adapt it for the lesion depth or volume using an algorithm implemented in the software, as described hereinbelow. The software thus provides real-time feedback that may support clinical decision making.

Again, as compared to the known art, the present embodiments use a different cooling technology, cold liquid nitrogen streaming as opposed to cold gaseous, supercritical or near-critical nitrogen streaming.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

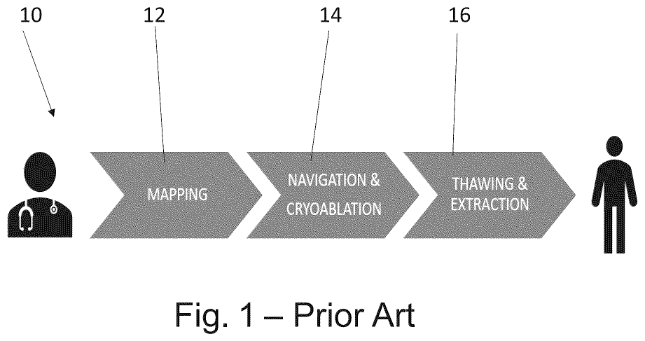

Referring now to the drawings, FIG. 1 is a simplified flow chart that illustrates a procedure 10 for cardiac cryoablation by catheterization. The process involves imaging and/or mapping 12 of the patient. Imaging includes CT (computed tomography), MRI (Magnetic Resonance Imaging), Roentgen, or Fluoroscopy. Specifically, an electro-anatomic mapping procedure is carried out. A mapping device may be specified in the user manual of the cryoablation device as appropriate. Mapping may be followed by navigation to bring the catheter and cryoablation tip to the cryoablation site. Optionally, the cryoablation catheter may have electro-mapping components, as will be discussed in greater detail below, so the mapping 12 and the navigation 14 may be carried out in a single action. Once the cryoablation catheter is in the correct location the user may release the tip of the catheter from the sheath to the desired diameter, for example using a mechanical mechanism that is integrated in the catheter, then migrate the tip to the tissue and choose on the graphical user interface the available option to start freezing. The procedure may be stopped at any time by the user. The procedure may be considered complete at some point in time as decided by the operator and the decision may be based on imaging techniques such as Fluoroscopy, and/or decision-supporting software integrated in the cryoablation device, described herein.

The completed procedure is followed by thawing and extraction 16. Prior to the catheter extraction the operator may wait for thawing of ice created by the procedure. The thawing 16 may be natural (un-forced) or forced. Forced thawing is an optional feature of the cryoablation system and may be enhanced by supply of warm gas to the tip or by electrical warming of the tip. Based on sensor readings, the software may indicate that the tip may be moved to the next location or folded back into the catheter, and the catheter may be extracted.

The embodiments may be suitable for, but not limited to, for treating cardiac disorders (arrythmias) such as: Atrial fibrillation, Atrial flutter, SVT, Atrial tachycardia, Ventricular fibrillation, Ventricular tachycardia, and Premature Ventricular Condition or Complex. It will be appreciated that only a patient referred by a cardiologist or electro-physiologist who is certified for the purpose may undergo the cryoablation procedure. Only the interventional cardiologist or electro-physiologist (end user), certified and instructed to use the device, may perform the procedure, and the term “user” herein may be construed accordingly.

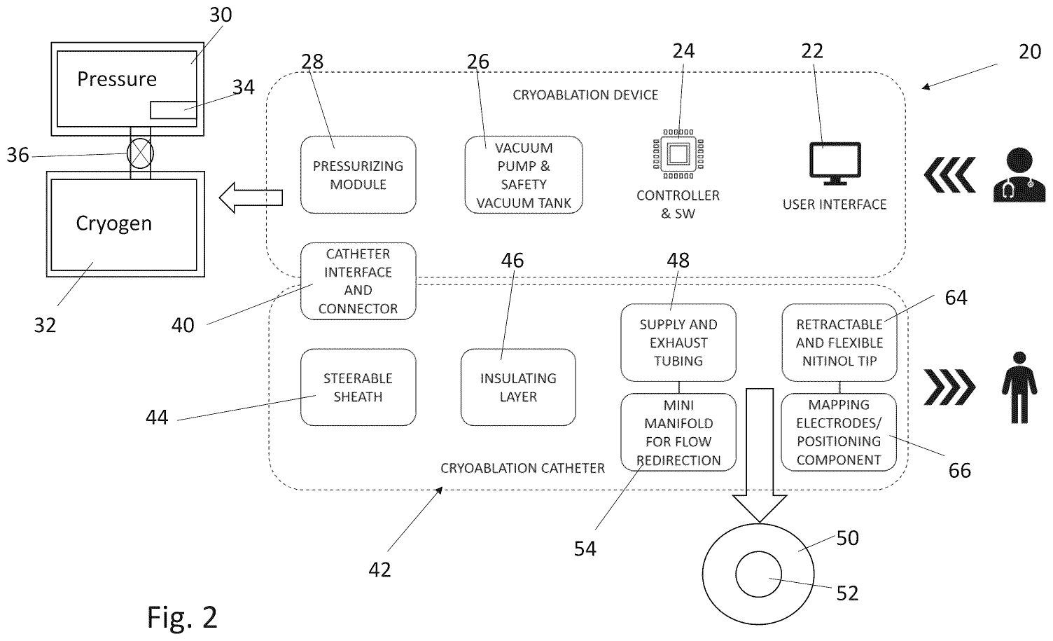

Reference is now made to FIG. 2 which illustrates the modules of a cryoablation device 20 according to the present embodiments.

User interface 22 may consist of an off-the-shelf medical grade PC or similar computing device having a GUI which is simple and user-friendly. Optionally, the GUI may allow real-time sensor readings and the sensors may be customized. For example, the display may be set to show total procedure time, LN2 tank level, Calculated ablation volume/depth, etc.

Controller 24 may be an off-the-shelf controller or an in-house developed field programmable gate array FPGA. The controller may have sufficient inputs/outputs and interfaces to service all the electrically switchable components of the system such as sensors, valves, heaters, relays, indicators, etc. The controller may optionally be integrated with the user interface into a single computing device.

Software for the controller 24 may manage the system and the GUI. The development environment may depend on the chosen controller, for example Labview, C, etc.). Safety aspects may be resolved both by software and by hardware components.

The cryoablation device may comprise an insulation and safety device. The device may include a vacuum pump & safety vacuum tank 26. The cryoablation catheter 42 may require an insulation layer 46, and the vacuum for insulation layer 46 may be actively supplied by the vacuum pump 26. The safety vacuum tank 26 may be integrated in the vacuum tubing system and may act as a buffer to prevent exposure of the patient to the cryogenic material. In the case of leakage of the cryogenic material to the outer layers (insulation and sheath).

The supply device may further include a pressurizing module 28. The module may include a pressurizing system whose design is discussed herein. The pressurizing module may comprise two well-insulated separate tanks 30 and 32, both filled with liquid nitrogen (LN2). The first of the two tanks may act as a pressure source and the second as a cryogen source for the cryoablation. The pressure may be increased by operating a heating unit 34 inside the first tank or by compressor, connected pneumatically to that tank.

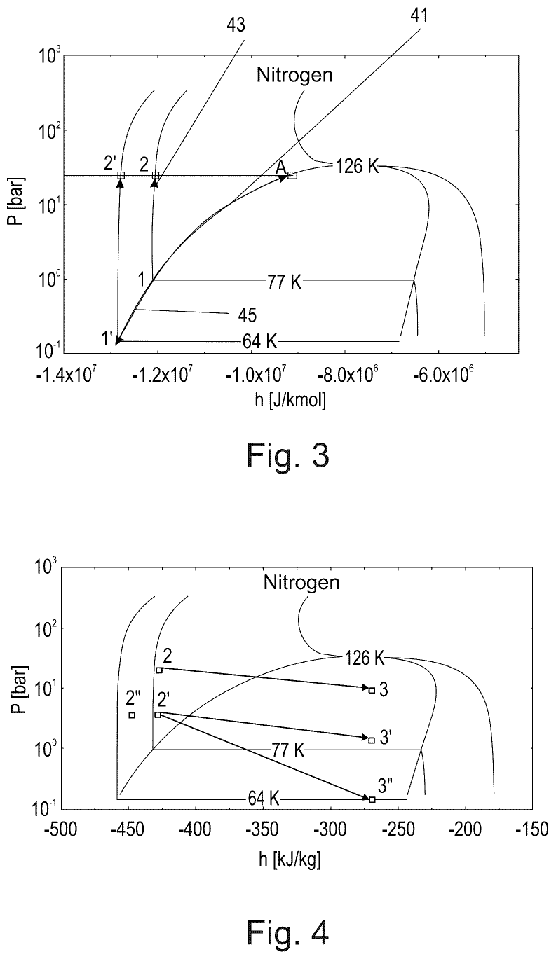

Reference is now made to FIG. 3, which is a graph showing pressure characteristics as the pressurizing module is operated. During heating, the pressure follows section 1—A of curve 41 in FIG. 3. The second tank is connected pneumatically to the first by a tube and a valve 36. The valve is opened when the procedure starts, and pressure propagates to the second tank as indicated by section 1-2 of curve 43 in FIG. 3.

Optionally, also during the pre-procedure stage the system may lower the temperature of the cryogenic material in the second tank to 64 K (˜−210° C.) using vacuum suction represented by process 1-1′ (curve 45) in FIG. 3.

The configuration of the pressurizing module and its operation according to FIG. 3 allows the cryogenic material supplied to the system to stay in a supercooled state. Thus, the LN2 may be kept at 20 bar, and at 77 K (point 2) (or 64 K (point 2′) as described in greater detail hereinbelow) and this enables the module to provide an additional energy capacity to the process by utilizing enthalpy of the supercooling (the horizontal distance between the points 2 and A in FIG. 3).

The inlet pressure, at point 2 or 2′ may be controlled in accordance with the exhaust temperature or steam quality which may be measured by a mass flux sensor on the exhaust cryogenic material. That is to say a condition is provided that the exhaust temperature and pressure fit the bounds of boiling fluid, and the software may decrease the inlet pressure accordingly. As the inlet pressure drops the latent heat of the coolant rises, so the cooling efficiency of the system rises. For example, in FIG. 4 the process 2′-3′ has a greater latent heat than 2-3.

The cryoablation device is connected to the cryoablation catheter 42 via a catheter interface and connector 40. The catheter interface and connector may be off-the-shelf or specifically developed components and may provide a sealed and safe connection between the catheter 42 and the device 20. The cryoablation catheter 42 may include a steerable sheath 44. Again, the steerable sheath may be an off-the-shelf or specially developed component and may include an integrated mechanical steering mechanism. The sheath provides for the navigation of the catheter and tubing to the site of the ablation, also provides tip hosting and tip release and further provides for safe retraction at the end of the procedure.

An insulating layer 46 may be provided as a persistent sleeve of foamed polymer or as a vacuum gap jacket actively pulled by Vacuum Pump 26 as already described.

Supply and exhaust tubing 48 may consist of two concentric tubes 50 and 52 of reinforced polymer or metal or alloy, e.g. stainless-steel thin wall tubes or nitinol tubes. On the one hand the wall thickness of the tubes may be made as thin as possible to prove flexibility, however the thickness must be sufficient to withstand the work pressure at different work stages and temperatures. Thus, pressure may range from vacuum to 20 bar or above over the course of the procedure and the temperature may range from minus 212° C. to plus 50° C. The supply tube may be the inner 52 of the two concentric tubes and the outer tube 50 may carry the exhaust and provide insulation for the inner tube.

In theory, it is possible to connect a strong enough vacuum pump to the exhaust tube to decrease the outlet pressure, say from point 3′ to 3″ in FIG. 4. Points 3, 3′ and 3″ shown in FIG. 4, represent properties of the cryogen after it passes through the heat exchanger, namely the operating tip. If the pressure in the exhaust tube drops to near 1 bar or below (point 3″ in FIG. 4), the temperature of the boiling liquid in it may in fact be even lower than in the supply tube (points 2 and 2′ in FIG. 4). In this way, the exhaust tube cools the supply tube (from point 2′ to 2″), reaching additional cooling enthalpy and increasing the efficiency of the process.

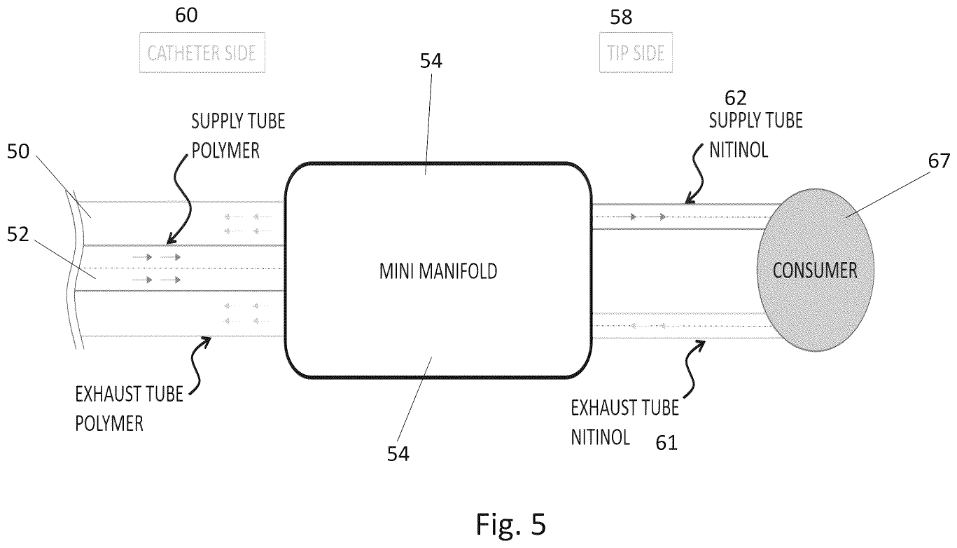

The embodiment may have a mini manifold 54 which is designed for flow redirection. The mini manifold is provided as part of the catheter tubing and acts as a connector between the tip side 58 and the tubing side 60, as shown in FIG. 5. The manifold may direct the exhaust flow from the tip exhaust tube 61 to the outer concentric tube. In addition, the manifold may direct the supply cryogen from the supply tube 50 to the tip supply tube 62.

Returning now to FIG. 2 and the cryoablation catheter 42 further comprises a retractable and flexible tip 64. The tip may be constructed using shape-memory material, such as nitinol. The purpose of the tip in cryoablation, is to transfer heat from the consumer 67, that is the tissue being treated, to the cryogenic fluid. Thus, the tip may have or may not have inner heat-exchanging components or such features as ribs, inserts etc. Prior to the cooling stage, that is during preparation and navigation, and after cooling, during retraction etc., the tip is folded inside the sheath. Once the catheter is in the final position before cooling, the user may release the tip from the sheath using a mechanical mechanism integrated in the sheath.

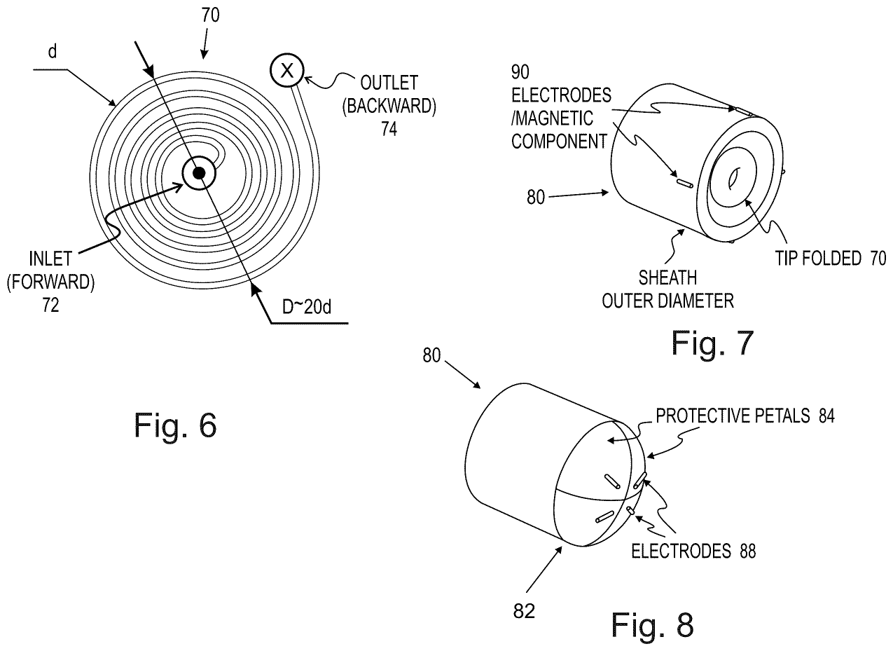

Referring now to FIG. 6, the tip may be shaped in various ways according to the specific application. For example, for VT ablation tip 70 as shown in FIG. 6 may have a spiral shape. The nitinol tube may thus have a relatively small diameter, say ϕ0.1-1 mm, but may nevertheless produce a large footprint in terms of surface area say ˜3 cm². It is desirable to ablate an area as large as possible in one go to reduce the number of treated locations, and thus, reduce the overall procedure duration and risk of complications so as to achieve a successful procedure. The cryogenic material, in this case liquid nitrogen, is provided to inlet 72 via the manifold 54, from supply tube 52, and exits via outlet 74 where it may be directed, again by the manifold 54, to the exhaust tube 50.

In an embodiment, the final footprint of the spiral-shape tip may be controlled by use of a release mechanism. Thus, one may use the same catheter to make ablations of 0.3 cm² or 3 cm².

In a further embodiment, a thin polymer coating may be applied to the tip, partially or fully, to prevent or reduce heat transfer from the surroundings of the treated area (e.g. blood flow, chordae tendineae) and for easier tip detaching from the cold treated area.

Returning again to FIG. 2, mapping electrodes or a positioning component may be provided, both are off-the-shelf components. That is to say, in order to correctly locate the catheter at the ablation location prior to cooling it is necessary to use a mapping device. Any component to used may be integrated at the distal end of the sheath—see FIGS. 7 and 8, so that the user may lock the steering mechanism when the sheath end is in the proper location.

Reference is now made to FIGS. 7 and 8, which illustrate optional general structures of the distal end 80 of a catheter according to the present embodiments.

The distal end 80 of the sheath may comprise a plug 82 made up of petals 84 as shown in FIG. 8, or may not as shown in FIG. 7. The petals are made of biocompatible metal or polymer. The purpose of the plug is to provide a streamlined shape so as to prevent blood vessels from being damaged through the navigation stage when the catheter navigates through blood vessels to the cryoablation site. The petals may act as a check valve and open only from the inside while releasing the tip 70. The Embodiment comprising tissue impedance electrodes 88 may be used as shown in FIG. 8, while, separately, the embodiment comprising magnetic positioning component 90 may be used as shown in FIG. 7, or vice versa.

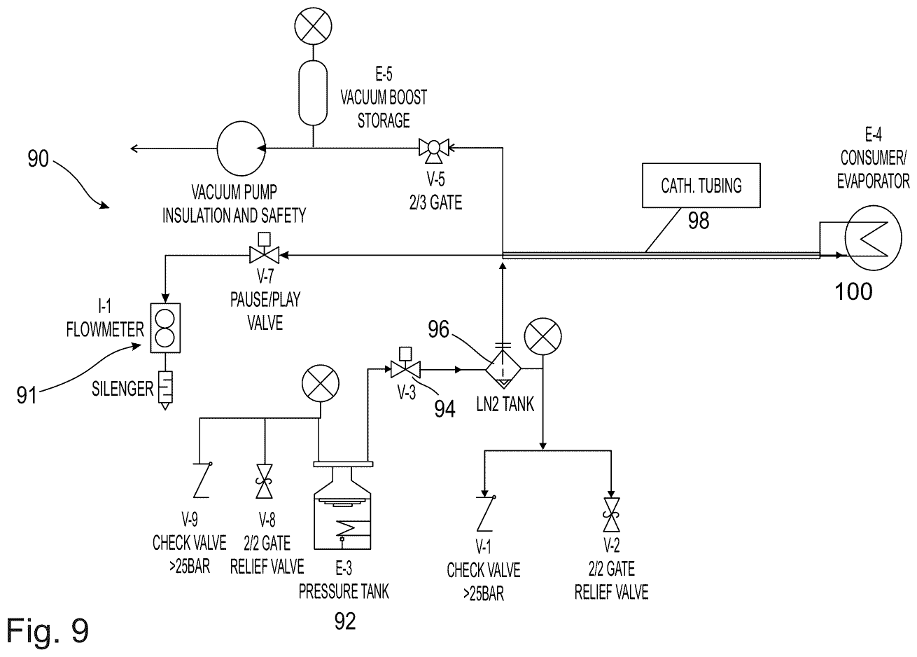

Reference is made to FIG. 9, which is a simplified diagram illustrating the pneumatic circuit 90 of the present embodiments. Pressure is provided by pressure tank 92 which is connected by valve 94 to LN2 tank 96. The LN2 is supplied under pressure from the pressure tank to catheter tubing 98 and from there to the tip 100 where it is consumed. Exhaust returns through the catheter tubing as described hereinabove.

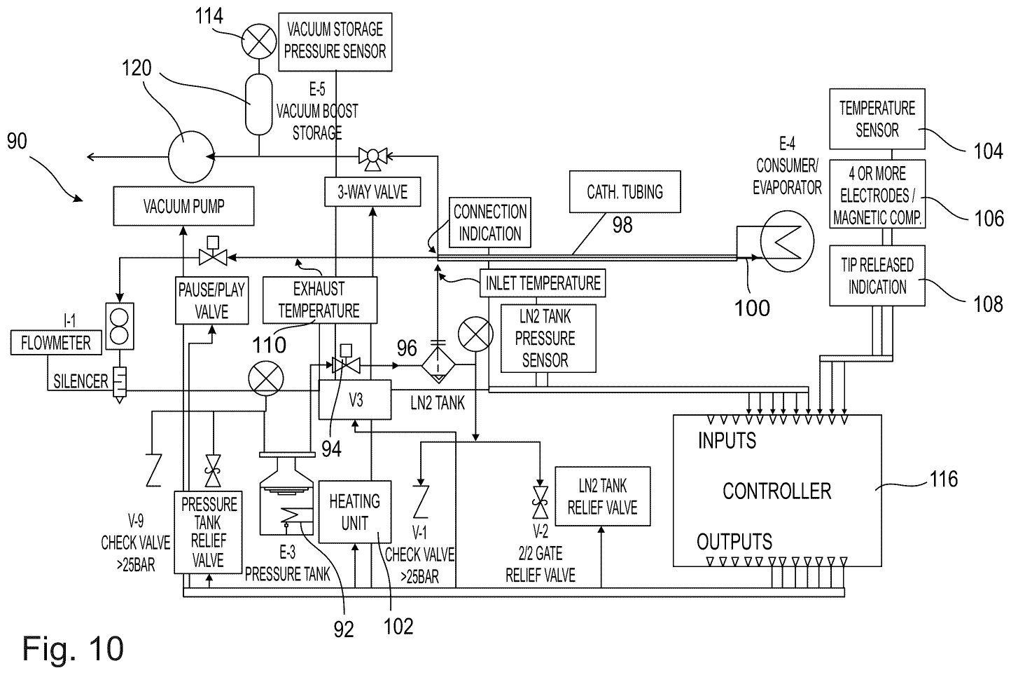

Reference is now made to FIG. 10, which illustrates a control scheme for the present embodiments. The control scheme is overlaid over the pneumatic scheme 90 and the same reference numerals are used for the parts already described in FIG. 9. Pressure is provided by pressure tank 92 and pressure is changed by means of heating using heater 102. The pressure tank 92 is connected by valve 94 to LN2 tank 96 and the valve may be controllably operated by the control system. The LN2 is supplied under pressure from the pressure tank to catheter tubing 98, where the connection quality and the inlet temperature are monitored. The LN2 is supplied via the tubing 98 to the tip 100 where it is consumed. At the tip itself, temperature may be monitored by temperature sensor 104. The electrodes may be operated 106 and a tip release indication 108 may be provided. Exhaust returns through the catheter tubing as described hereinabove. Exhaust temperature may be monitored using exhaust temperature sensor 110. All the controlling inputs, outputs and sensors are connected to controller 116.

Reference numeral 120 indicates vacuum pump and storage, which are provided in parallel to the exhaust line and keep the insulation layer under necessary vacuum, and, optionally, may lower pressure at the tip outlet, for which an additional valve may be required. A vacuum storage pressure sensor 114 may be provided.

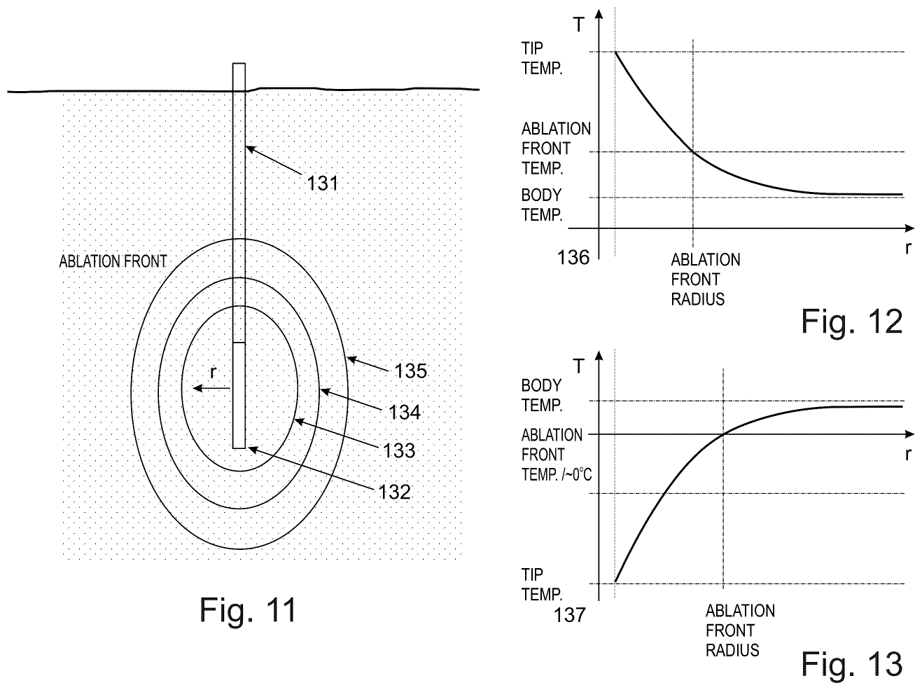

A decision support tool, as mentioned hereinabove, may be provided for use with the above-discussed embodiments. Such a support tool relies on an algorithm that evaluates utilized energy and compares the utilized energy to the energy inherent in a particular volume of human tissue. Such an energy approach or algorithm is suitable either for heating or cooling ablations. Referring now to FIG. 11, percutaneous catheter, or probe 131 has an active tip 132 which is placed in the required tissue. Once the procedure begins the temperature at the tip's surface changes accordingly to the application, heating is represented at the upper graph 136, FIG. 12, and cooling is represented at the lower graph 137, FIG. 13. Lines 133, 134 and 135 represent asymptotic fronts of ablation that depend on the power of the device, low, moderate, and high power increasing towards the tip. The higher power available to the present embodiments, as opposed to the existing art, has the capability to achieve a greater ablation front radius (for example as shown on graphs 136 and 137), which may further depend on the procedure duration. Multiplying power by duration gives the total amount of energy supplied by the system to the treated environment. Knowing tissue physical properties such as density, heat capacity and heat conductivity, it is possible to determine how much energy is required to change the temperature of the desired volume from the initial 37° C. to the desired ablation temperature, as shown on graphs 136 and 137. The embodiment may have an algorithm embedded in the GUI that may compare in real-time the ratio of utilized to desired amount of energy and provide an indication of when the desired amount is reached. The actual ablation front radius when the system indicates that the required energy level has been reached may be smaller than desired, but may keep growing due to the accumulated energy even after the tip activation stop. Additional corrections of the energy comparison may be made according to the treated environment. Thus, for example in radiology in liver ablation one may prefer achieving 110% of the desired amount of energy, while in cardiology just 90% of the desired energy may be utilized to ensure the safety of the procedure.

While the power evaluation in heating applications is relatively simple and requires knowing only the active tip's electrical power consumption, the power evaluation for cooling applications necessitates monitoring the thermodynamic values of the coolant. For the present embodiments, cryogenic flowmeter 91 in FIG. 9 may be placed at the outlet line before the coolant discharge to the environment, and sense volumetric or mass flow rate of both phases, liquid and gaseous, of the coolant. Knowing initial and final temperature and pressure of the coolant, flow rate and vapor quality, allows coolant enthalpy change evaluation. The enthalpy change value represents the total energy loss of the coolant passing through the system and the active tip. It is thus reasonable to evaluate the nominal system cooling energy consumption without ablation load to receive the actual cooling power of the tip.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment and the present description is to be construed as if such embodiments are explicitly set forth herein. Conversely, various features of the invention, which are, for brevity, described in the context of a single embodiment, may also be provided separately or in any suitable subcombination or may be suitable as a modification for any other described embodiment of the invention and the present description is to be construed as if such separate embodiments, subcombinations and modified embodiments are explicitly set forth herein. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. Apparatus for cryoablation comprising:
a supply unit configured to provide super-cooled liquid nitrogen for streaming;
a cryoablation tip for use at a cryoablation site, the tip configured to receive said super-cooled or boiling liquid nitrogen and to exchange heat using said liquid nitrogen, thereby to carry out ablation.

2. The apparatus of claim 1, wherein said supply unit comprises a pressure tank and a liquid nitrogen tank, the pressure tank being connected to the liquid nitrogen tank and being controllable to provide a pressure to maintain the liquid nitrogen as a liquid at boiling point or below, thereby to take advantage of latent heat to prevent changes of temperature of said liquid nitrogen.

3. The apparatus of claim 2, wherein the liquid nitrogen tank comprises two layers of vacuum as insulation.

4. The apparatus of claim 2, comprising catheter tubing to connect between said tip and said liquid nitrogen tank, the catheter tubing comprising an inner tube and a concentrically located outer tube, the inner tube for supplying liquid nitrogen to said tip and the outer tube for carrying exhaust and for insulating said inner tube.

5. The apparatus of claim 4, further comprising a manifold between said catheter tubing and said tip, the manifold configured to connect the inner tube to provide said liquid nitrogen to an inlet of said tip, and to connect the outer tube to an outlet of said tip.

6. The apparatus of claim 1, further comprising a sheath, said sheath having a proximal end and a distal end, the distal end containing said tip, the sheath being controllably steerable to enable said distal end to reach a cryoablation site.

7. The apparatus of claim 6, wherein said sheath comprises a closure over said tip, the closure being controllably openable on reaching said cryoablation site to reveal said tip.

8. The apparatus of claim 6, wherein said sheath comprises electrodes for said controllable steering of said sheath.

9. The apparatus of claim 1, wherein said tip comprises a hollow tube, the hollow tube having an inlet for inflow of said liquid nitrogen and an outlet for outflow of said liquid nitrogen after use as exhaust.

10. The apparatus of claim 9, wherein said hollow tube is arranged in a coil.

11. The apparatus of claim 9, wherein said tip is made of a material having a shape memory.

12. The apparatus of claim 11, further comprising a controllable release mechanism to release said tip in different shapes for different energy release requirements.

13. The apparatus of claim 1, configured to control pressure at a tip inlet in order to maintain said liquid nitrogen as a boiling liquid.

14. The apparatus of claim 1, comprising a vacuum source connected to an outlet of said tip.

15. The apparatus of claim 1, further comprising a control module to regulate an amount of energy released at said tip by calculating an amount of energy needed by a defined volume of tissue.

16. Method for cryoablation comprising:
providing super-cooled liquid nitrogen for streaming;
streaming said super-cooled liquid nitrogen to reach a cryoablation tip at lower than boiling temperature for use at a cryoablation site, thereby to carry out ablation.

17. The method of claim 16, comprising monitoring an amount of energy released at said tip and comparing said amount with energy needed by a defined volume of tissue.
